(19) United States
(12) Patent Application Publication
(10) Pub. No.: US 2026/0036538 A1
(43) Pub. Date: Feb. 5, 2026

(54) FOOD MANAGEMENT SYSTEM AND METHOD OF CONTROLLING THE SAME

(71) Applicant: Samsung Electronics Co., Ltd., Suwon-si (KR)

(72) Inventors: Jaeyong CHO, Suwon-si (KR); Junhyeong PARK, Suwon-si (KR); Daehyun KIM, Suwon-si (KR); Sungbum PARK, Suwon-si (KR)

(21) Appl. No.: 19/343,070

(22) Filed: Sep. 29, 2025

Related U.S. Application Data
(63) Continuation of application No. PCT/KR2025/011496, filed on Aug. 1, 2025.

(30) Foreign Application Priority Data
Aug. 2, 2024 (KR) 10-2024-0103428

(51) Int. Cl. G01N 27/02; A23B 2/00; A23B 2/60; A23B 2/82; A23B 4/015; A23B 4/07; G01N 33/02

(52) U.S. Cl. CPC G01N 27/02; A23B 2/001; A23B 2/003; A23B 2/60; A23B 2/82; A23B 4/015; A23B 4/07; G01N 33/02

(57) ABSTRACT

A food management system is provided. The food management system includes a plurality of electrodes arranged to be spaced apart from each other with food therebetween, a frequency modulator configured to adjust a frequency of power supplied to the plurality of electrodes, memory, comprising one or more storage media, storing instructions, and at least one processor communicatively coupled to the plurality of electrodes, the frequency modulator, and the memory, wherein instructions, when executed by the at least one processor individually or collectively, cause the food management system to control the frequency modulator such that power at each of a plurality of frequencies is sequentially supplied to the plurality of electrodes, determine a resonance frequency based on an impedance of the food at each frequency, determine a state of the food based on the resonance frequency of the food, and perform an operation corresponding to the determined state of the food.

### CROSS-REFERENCE TO RELATED APPLICATION(S)

This application is a continuation application, claiming priority under 35 U.S.C. § 365 (c), of an International application No. PCT/KR2025/011496, filed on Aug. 1, 2025, which is based on and claims the benefit of a Korean patent application number 10-2024-0103428, filed on Aug. 2, 2024, in the Korean Intellectual Property Office, the disclosure of which is incorporated by reference herein in its entirety.

### TECHNICAL FIELD

The disclosure relates to a food management system capable of managing food and a method of controlling the same.

### BACKGROUND ART

The quality of food may vary depending on the storage method and storage period. For example, meat, which is a type of livestock products, provides an environment in which microorganisms may easily proliferate. Among beef, pork, and chicken, chicken meat in particular spoils easily and therefore requires careful management during storage and distribution processes.

The conventional food storage devices, such as refrigerators may allow food spoilage to occur when users fail to recognize or forget the expiration date of stored food, resulting in prolonged storage. Storage may be particularly difficult for foods that have a short expiration date and spoil easily, such as meat.

The above information is presented as background information only to assist with an understanding of the disclosure. No determination has been made, and no assertion is made, as to whether any of the above might be applicable as prior art with regard to the disclosure.

### DISCLOSURE

### Technical Problem

Aspects of the disclosure are to address at least the above-mentioned problems and/or disadvantages and to provide at least the advantages described below. Accordingly, an aspect of the disclosure is to provide a food management system and a method of controlling the same capable of extending the storage period of food by automatically performing sterilization after identifying the state of food, such as the degree of spoilage.

Another aspect of the disclosure is to provide a food management system and a method of controlling the same in which the food state identification and sterilization are automatically performed and thus user convenience may be enhanced by eliminating the need for users to directly manage food to prevent spoilage.

Another aspect of the disclosure is to provide a food management system and a method of controlling the same allowing appropriate cooking to be achieved by identifying a cooking state of food, such as the degree of thawing, and adjusting power accordingly.

Additional aspects will be set forth in part in the description which follows and, in part, will be apparent from the description, or may be learned by practice of the presented embodiments.

### Technical Solution

In accordance with an aspect of the disclosure, a food management system is provided. The food management system includes a plurality of electrodes arranged to be spaced apart from each other with food therebetween, a frequency modulator configured to adjust a frequency of power supplied to the plurality of electrodes, memory, including one or more storage media, storing instructions, and at least one processor communicatively coupled to the plurality of electrodes, the frequency modulator and the memory, wherein the instructions, when executed by the at least one processor individually or collectively, cause the food management system to control the frequency modulator such that power at each of a plurality of frequencies may be sequentially supplied to the plurality of electrodes, determine a resonance frequency based on an impedance of the food at each frequency, determine a state of the food based on the resonance frequency of the food, and perform an operation corresponding to the determined state of the food.

In accordance with another aspect of the disclosure, a method of controlling a food management system is provided. The method includes a plurality of electrodes arranged to be spaced apart from each other with food therebetween and a frequency modulator configured to adjust a frequency of power supplied to the plurality of electrodes may include controlling the frequency modulator such that power at each of a plurality of frequencies is sequentially supplied to the plurality of electrodes, determining a resonance frequency based on an impedance of the food at each frequency, determining a state of the food based on the resonance frequency of the food, and performing an operation corresponding to the determined state of the food.

In accordance with another aspect of the disclosure, one or more non-transitory computer-readable storage media storing one or more computer programs including computer-executable instructions that, when executed by one or more processors of an electronic device individually or collectively, cause the electronic device to perform operations, the electronic device communicative coupled to a plurality of electrodes arranged to be spaced apart from each other with food therebetween and a frequency modulator configured to adjust a frequency of power supplied to the plurality of electrodes, are provided. The operations include controlling, by the electronic device, the frequency modulator such that power at each of a plurality of frequencies is sequentially supplied to the plurality of electrodes, determining, by the electronic device, a resonance frequency based on an impedance of the food at each frequency, determining, by the electronic device, a state of the food based on the resonance frequency of the food, and performing, by the electronic device, an operation corresponding to the determined state of the food.

Other aspects, advantages, and salient features of the disclosure will become apparent to those skilled in the art from the following detailed description, which, taken in conjunction with the annexed drawings, discloses various embodiments of the disclosure.

### DESCRIPTION OF DRAWINGS

The above and other aspects, features, and advantages of certain embodiments of the disclosure will be more apparent from the following description taken in conjunction with the accompanying drawings, in which:

FIG. 1 is a control block diagram illustrating a food storage system according to an embodiment of the disclosure;

FIG. 2 is a diagram illustrating a structure of a food storage system according to an embodiment of the disclosure;

FIG. 3 is a flowchart illustrating a method of identifying a state of food in a food storage system according to an embodiment of the disclosure;

FIG. 4 is a diagram illustrating irradiation of a near field (NF) magnetic field to food according to an embodiment of the disclosure;

FIG. 5 is a diagram for describing determination of resonance frequency according to an embodiment of the disclosure;

FIG. 6 is a flowchart showing a sterilization process according to a state of food in a food storage system according to an embodiment of the disclosure;

FIG. 7 is a diagram for describing determining a resonance frequency of food a plurality of times according to an embodiment of the disclosure;

FIG. 8A is a diagram illustrating adjusting the sterilization intensity and time according to an embodiment of the disclosure;

FIG. 8B is a diagram illustrating adjusting a sterilization intensity and time according to an embodiment of the disclosure;

FIG. 9 is a diagram for describing a degree of food spoilage depending on food storage environment according to an embodiment of the disclosure;

FIG. 10 is a flowchart for describing a process of determining whether food has been introduced according to an embodiment of the disclosure;

FIG. 11A is a diagram illustrating a plurality of sub-electrodes included in each of a plurality of electrodes according to an embodiment of the disclosure;

FIG. 11B is a diagram illustrating a plurality of sub-electrodes included in each of a plurality of electrodes according to an embodiment of the disclosure;

FIG. 11C is a diagram illustrating a plurality of sub-electrodes included in each of a plurality of electrodes according to an embodiment of the disclosure;

FIG. 12 is a diagram illustrating activation of some of a plurality of sub-electrodes according to an embodiment of the disclosure;

FIG. 13A is a diagram illustrating a method of determining an impedance using a plurality of sub-electrodes according to an embodiment of the disclosure;

FIG. 13B is a diagram illustrating a method of determining an impedance using a plurality of sub-electrodes according to an embodiment of the disclosure;

FIG. 13C is a diagram illustrating a method of determining an impedance using a plurality of sub-electrodes according to an embodiment of the disclosure;

FIG. 14A is a diagram for describing normal pH ranges of food and changes in pH over time according to an embodiment of the disclosure;

FIG. 14B is a diagram for describing normal pH ranges of food and changes in pH over time according to an embodiment of the disclosure;

FIG. 15 is a flowchart showing a method of identifying a food cooking state in a food storage system according to another embodiment of the disclosure; and

FIG. 16 is a diagram illustrating provision of a notification to a user and the like according to an embodiment of the disclosure.

The same reference numerals are used to represent the same elements throughout the drawings.

### MODES OF THE INVENTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Various embodiments of the disclosure and terms used therein are not intended to limit the technical features described in this document to specific embodiments, and should be understood to include various modifications, equivalents, or substitutes of the corresponding embodiments.

In connection with the description of the drawings, similar reference numerals may be used for similar or related components.

In this document, phrases, such as “A or B”, “at least one of A and B”, “at least one of A or B,” “A, B or C,” “at least one of A, B and C,” and “at least one of A, B, or C”, may include any one or all possible combinations of items listed together in the corresponding phrase among the phrases.

As used herein, the term “and/or” includes any and all combinations of one or more of associated listed items.

Terms, such as “1st”, “2nd”, “primary”, or “secondary” may be used simply to distinguish a component from other components, without limiting the component in other aspects (e.g., importance or order).

Further, as used in the disclosure, the terms “front”, “rear”, “top”, “bottom”, “side”, “left”, “right”, “upper”, “lower”, and the like are defined with reference to the drawings, and are not intended to limit the shape and position of each component.

It will be understood that when the terms “includes,” “comprises,” “including,” and/or “comprising,” when used in this specification, specify the presence of stated features, figures, steps, operations, components, members, or combinations thereof, but do not preclude the presence or addition of one or more other features, figures, steps, operations, components, members, or combinations thereof.

It will be understood that when a certain component is referred to as being “connected to”, “coupled to”, “supported by” or “in contact with” another component, it may be directly or indirectly connected to, coupled to, supported by, or in contact with the other component. When a component is indirectly connected to, coupled to, supported by, or in contact with another component, it may be connected to, coupled to, supported by, or in contact with the other component through a third component.

It will also be understood that when a component is referred to as being “on” another component, it may be directly on the other component or intervening components may also be present.

A refrigerator according to an embodiment of the disclosure may include a main body.

The main body may include an inner case, an outer case disposed outside the inner case, and an insulation between the inner case and the outer case.

The “inner case” may include at least one of a case, a plate, a panel, and a liner forming the storage compartment. The inner case may be formed as a single body, or may be formed by assembling a plurality of plates. The “outer case” may form an outer appearance of the main body, and may be coupled to the outside of the inner case such that an insulation is placed between the inner case and the outer case.

The insulation may insulate inside of a storage compartment from outside of the storage compartment to maintain inside temperature of the storage compartment at appropriate temperature without being influenced by an external environment of the storage compartment. According to an embodiment of the disclosure, the insulation may include a foaming insulation. The foaming insulation may be formed by injecting and foaming a urethane form, in which polyurethane and a foaming agent are mixed, between the inner case and the outer case.

According to an embodiment of the disclosure, the insulation may include a vacuum insulation in addition to a foaming insulation, or may be configured only with a vacuum insulation instead of a forming insulation. The vacuum insulation may include a core material and an envelope that accommodates the core material and seals the interior at a vacuum or a pressure close to vacuum. However, the insulation is not limited to the foaming insulation or vacuum insulation described above, and may include various materials that may be used for insulation.

The “storage compartment” may include a space defined by the inner case. The storage compartment may further include the inner case defining a space corresponding to the storage compartment. The storage compartment may store a variety of items, such as foods, medicines, cosmetics, and the like, and the storage compartment may be formed to be open on at least one side for storing or removing items.

The refrigerator may include one or more storage compartments. In a case in which two or more storage compartments are formed in the refrigerator, the respective storage compartments may have different purposes of use, and may be maintained at different temperature. To this end, the storage compartments may be partitioned by a partition wall including an insulation.

The storage compartment may be maintained within an appropriate temperature range according to a purpose of use, and include a “refrigerating compartment”, a “freezing compartment”, and a “temperature conversion compartment” according to purposes of use and/or temperature ranges. The refrigerating compartment may be maintained at appropriate temperature to keep food refrigerating, and the freezing compartment may be maintained at appropriate temperature to keep food frozen. The “refrigerating” may be keeping food cold without freezing the food, and for example, the refrigerating compartment may be maintained within a range of 0 degrees Celsius to 7 degrees Celsius. The “freezing” may be freezing food or keeping food frozen, and for example, the freezing compartment may be maintained within a range of −20 degrees Celsius to −1 degrees Celsius. The temperature conversion compartment may be used as any one of a refrigerating compartment or a freezing compartment according to or regardless of a user's selection.

The storage compartment may also be called various other terms, such as “vegetable compartment (also referred to as room)”, “freshness compartment”, “cooling compartment”, and “ice-making compartment”, in addition to “refrigerating compartment”, “freezing compartment”, and “temperature conversion compartment”, and the terms, such as “refrigerating compartment”, “freezing compartment”, “temperature conversion compartment”, etc., as used below need to be understood to represent storage compartments having the corresponding purposes of use and the corresponding temperature ranges.

The refrigerator according to an embodiment of the disclosure may include at least one door configured to open or close the open side of the storage compartment. The respective doors may be provided to open and close one or more storage compartments, or a single door may be provided to open and close a plurality of storage compartments. The door may be rotatably or slidably mounted on the front of the main body.

The door may seal the storage compartment in a closed state. The door may include an insulation, like the main body, to insulate the storage compartment in the closed state.

According to an embodiment, the door may include an outer door plate forming the front surface of the door, an inner door plate forming the rear surface of the door and facing the storage compartment, an upper cap, a lower cap, and a door insulation provided therein.

A gasket may be provided on the edge of the inner door plate to seal the storage compartment by coming into close contact with the front surface of the main body when the door is closed. The inner door plate may include a dyke that protrudes rearward to allow a door basket for storing items to be fitted.

According to an embodiment, the door may include a door body and a front panel that is detachably coupled to the front of the door body and forms the front surface of the door. The door body may include an outer door plate that forms the front surface of the door body, an inner door plate that forms the rear surface of the door body and faces the storage compartment, an upper cap, a lower cap, and a door insulation provided therein.

The refrigerator may be classified as French Door Type, Side-by-side Type, Bottom Mounted Freezer (BMF), Top Mounted Freezer (TMF), or One Door Refrigerator depending on the arrangement of the doors and the storage compartments.

The refrigerator according to an embodiment of the disclosure may include a cold air supply device for supplying cold air to the storage compartment.

The cold air supply device may include a machine, an apparatus, an electronic device, and/or a combination system thereof, capable of generating cold air and guiding the cold air to cool the storage compartment.

According to an embodiment of the disclosure, the cold air supply device may generate cold air through a cooling cycle including compression, condensation, expansion, and evaporation processes of refrigerants. To this end, the cold air supply device may include a cooling cycle device having a compressor, a condenser, an expander, and an evaporator to drive the cooling cycle. According to an embodiment of the disclosure, the cold air supply device may include a semiconductor, such as a thermoelectric element. Thermoelectric element may cool the storage compartment by heating and cooling actions through the Peltier effect.

The refrigerator according to an embodiment of the disclosure may include a machine compartment where at least some components belonging to the cold air supply device are installed.

The machine compartment may be partitioned and insulated from the storage compartment to prevent heat generated from the components installed in the machine compartment from being transferred to the storage compartment. To dissipate heat from the components installed inside the machine compartment, the machine compartment may communicate with outside of the main body.

The refrigerator according to an embodiment of the disclosure may include a dispenser provided on the door to provide water and/or ice. The dispenser may be provided on the door to allow access by the user without opening the door.

The refrigerator according to an embodiment of the disclosure may include an ice-making device that produces ice. The ice-making device may include an ice-making tray that stores water, an ice-moving device that separates ice from the ice-making tray, and an ice-bucket that stores ice generated in the ice-making tray.

The refrigerator according to an embodiment of the disclosure may include a controller for controlling the refrigerator.

The controller may include memory for storing and/or memorizing data and/or programs for controlling the refrigerator, and a processor for outputting control signals for controlling the cold air supply device, etc. according to the programs and/or data memorized in the memory.

The memory may store or record various information, data, commands, programs, and the like necessary for operations of the refrigerator. The memory may store temporary data generated while generating control signals for controlling components included in the refrigerator. The memory may include at least one of volatile memory or non-volatile memory, or a combination thereof.

The processor may control the overall operation of the refrigerator. The processor may control the components of the refrigerator by executing programs stored in memory. The processor may include a separate neural processing unit (NPU) that performs an operation of an artificial intelligence (AI) model. In addition, the processor may include a central processing unit (CPU), a graphics processor (GPU), and the like. The processor may generate a control signal to control the operation of the cold air supply device. For example, the processor may receive temperature information of the storage compartment from a temperature sensor, and generate a cooling control signal for controlling an operation of the cold air supply device based on the temperature information of the storage compartment.

Furthermore, the processor may process a user input of a user interface and control an operation of the user interface according to the programs and/or data memorized/stored in the memory. The user interface may be provided using an input interface and an output interface. The processor may receive the user input from the user interface. In addition, the processor may transmit a display control signal and image data for displaying an image on the user interface to the user interface in response to the user input.

The processor and memory may be provided integrally or may be provided separately. The processor may include one or more processors. For example, the processor may include a main processor and at least one sub-processor. The memory may include one or more memories.

The refrigerator according to an embodiment of the disclosure may include a processor and memory for controlling all the components included in the refrigerator, and may include a plurality of processors and a plurality of memories for individually controlling the components of the refrigerator. For example, the refrigerator may include a processor and memory for controlling the operation of the cold air supply device according to an output of the temperature sensor. In addition, the refrigerator may be separately equipped with a processor and memory for controlling the operation of the user interface according to the user input.

A communication module may communicate with external devices, such as servers, mobile devices, and other home appliances via a nearby access point (AP). The AP may connect a local area network (LAN) to which a refrigerator or a user device is connected to a wide area network (WAN) to which a server is connected. The refrigerator or the user device may be connected to the server via the WAN.

The input interface may include keys, a touch screen, a microphone, and the like. The input interface may receive the user input and pass the received user input to the processor.

The output interface may include a display, a speaker, and the like. The output interface may output various notifications, messages, information, and the like generated by the processor.

The above description has illustrated a refrigerator as one example of the food management system 1. However, the food management system 1 is not limited to refrigerators and may include various devices capable of storing or cooking food.

It should be appreciated that the blocks in each flowchart and combinations of the flowcharts may be performed by one or more computer programs which include computer-executable instructions. The entirety of the one or more computer programs may be stored in a single memory device or the one or more computer programs may be divided with different portions stored in different multiple memory devices.

Any of the functions or operations described herein can be processed by one processor or a combination of processors. The one processor or the combination of processors is circuitry performing processing and includes circuitry like an application processor (AP, e.g., a central processing unit (CPU)), a communication processor (CP, e.g., a modem), a graphical processing unit (GPU), a neural processing unit (NPU) (e.g., an artificial intelligence (AI) chip), a wireless-fidelity (Wi-Fi) chip, a Bluetooth™ chip, a global positioning system (GPS) chip, a near field communication (NFC) chip, connectivity chips, a sensor controller, a touch controller, a finger-print sensor controller, a display drive integrated circuit (IC), an audio CODEC chip, a universal serial bus (USB) controller, a camera controller, an image processing IC, a microprocessor unit (MPU), a system on chip (SoC), an IC, or the like.

Hereinafter, the food management system according to various embodiments will be described in detail with reference to the accompanying drawings.

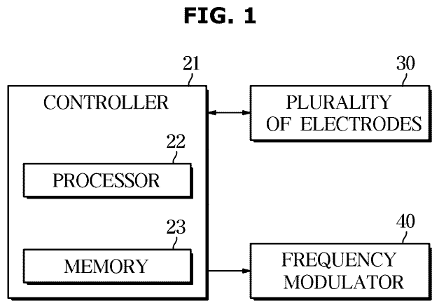

FIG. 1 is a control block diagram illustrating a food storage system according to an embodiment of the disclosure.

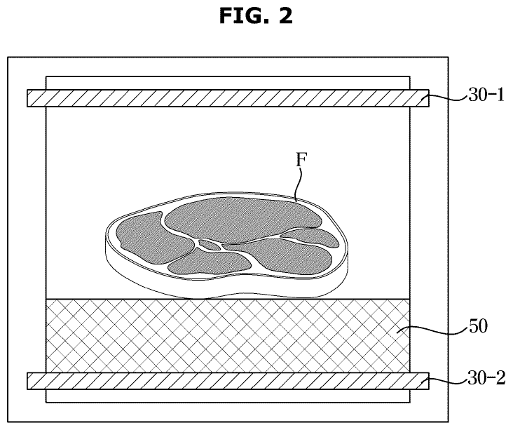

FIG. 2 is a diagram illustrating the structure of a food storage system according to an embodiment of the disclosure.

Referring to FIG. 1, the food storage system according to an embodiment may include a plurality of electrodes 30, a frequency modulator 40, and a controller 21. The controller 21 may include a processor 22 and the memory 23.

The plurality of electrodes 30 may generate a near field (NF) electric field to measure an impedance between electrodes. The plurality of electrodes 30 may be arranged to be spaced apart from each other with the food F in between to measure the impedance of the food F described below or to generate an electric field for sterilization of the food F.

For example, referring to FIG. 2, two electrodes may be arranged above and below each other with the food F in between. That is, the food F may be positioned between a first electrode 30-1 provided at an upper side of food and a second electrode 30-2 provided at a lower side of a seating portion 50.

Here, the seating portion 50 is a component for positioning the food F between the plurality of electrodes 30, and may be formed of Styrofoam, for example.

Referring to FIG. 2, the plurality of electrodes 30 are illustrated as being positioned above and below the food F as an example, the positions of the plurality of electrodes 30 are not limited thereto and may be provided at various positions capable of generating an electric field between the plurality of electrodes 30.

The frequency modulator 40 may adjust the frequency of power supplied to the plurality of electrodes 30. As will be described below, the frequency modulator 40 may adjust the frequency to measure impedances at various frequencies to determine a resonance frequency corresponding to food positioned between the plurality of electrodes 30.

The controller 21 may include the memory 23 that stores control programs and control data for controlling the plurality of electrodes 30, the frequency modulator 40, and the like, and the at least one processor 22 that generates control signals according to the control programs and the control data stored in the memory 23. The memory 23 and the processor 22 may be provided integrally or separately.

The memory 23 may store a table map and the like that represent the correlation between the pH of the food and the state of the food which will be described below, and may store programs and data for controlling the plurality of electrodes 30, the frequency modulator 40, and the like.

The memory 23 may include volatile memory such as static random access memory (S-RAM) and dynamic random access memory (D-RAM) for temporarily storing data. In addition, the memory 23 may include non-volatile memory such as read only memory (ROM), erasable programmable read only memory (EPROM), and electrically erasable programmable read only memory (EEPROM) for long-term storage of data.

The at least one processor 22 may control the frequency modulator 40 such that power at each of plurality of frequencies is sequentially supplied to the plurality of electrodes 30.

In addition, the at least one processor 22 may determine the resonance frequency based on impedances of the food F at each frequency.

The at least one processor 22 may determine the state of the food F based on the resonance frequency of the food and control performance of operations corresponding to the determined state of the food.

The following describes a process of determining the state of food and performing a sterilization operation.

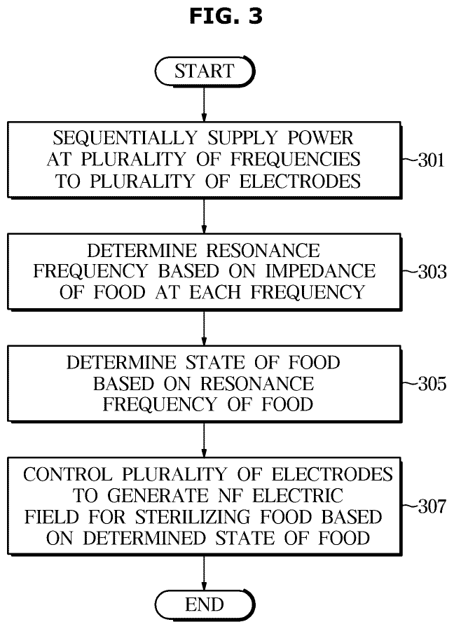

FIG. 3 is a flowchart illustrating a method of identifying a state of food in a food storage system according to an embodiment of the disclosure.

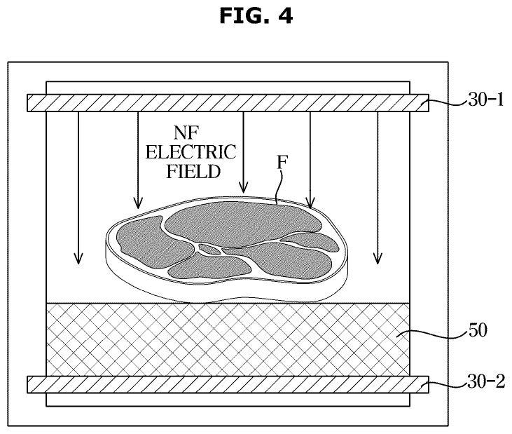

FIG. 4 is a diagram illustrating irradiation of a near field (NF) magnetic field to food according to an embodiment of the disclosure.

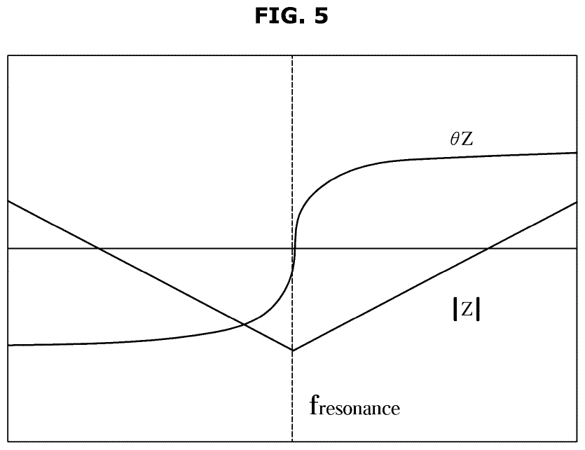

FIG. 5 is a diagram for describing determination of resonance frequency according to an embodiment of the disclosure.

To identify the state of the food F, for example, the degree of spoilage of the food F, the resonance frequency may be used. The resonance frequency increases as the food F spoils. Therefore, by periodically measuring such resonance frequency, the degree of food spoilage may be determined according to changes in the resonance frequency.

Referring to FIG. 3, the at least one processor 22 may control the frequency modulator 40 such that power at each of plurality of frequencies is supplied to the plurality of electrodes 30 as described above at operation 301.

The at least one processor 22 may determine the resonance frequency based on the impedance of the food F at each frequency at operation 303.

That is, power at each of various frequencies is supplied, and a frequency at which the current or voltage is maximized or the impedance is minimized among the plurality of frequencies may be determined.

Referring to FIG. 5, a frequency at which the impedance is minimized among the plurality of frequencies may be determined as the resonance frequency f_{resonance}.

The at least one processor 22 may determine the state of the food F based on the determined resonance frequency of the food at operation 305. For example, the degree of spoilage of the food F may be determined.

The at least one processor 22 may control the plurality of electrodes 30 to generate a NF electric field for sterilizing the food F based on the determined state of the food F at operation 307.

For example, referring to FIG. 4, a NF electric field may be generated from a first electrode 30-1 toward a second electrode 30-2 to sterilize food provided on the seating portion.

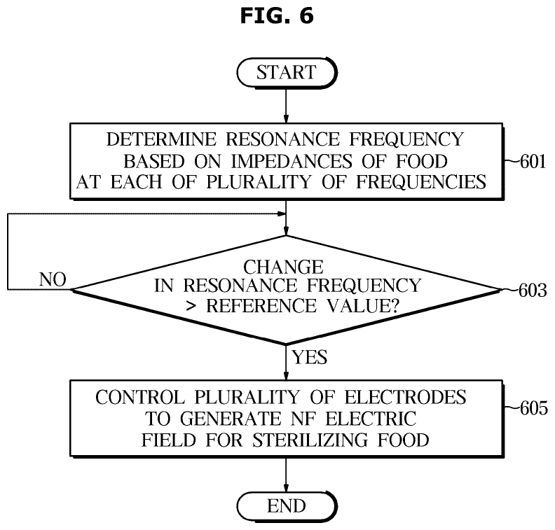

FIG. 6 is a flowchart showing a sterilization process according to the state of food in a food storage system according to an embodiment of the disclosure.

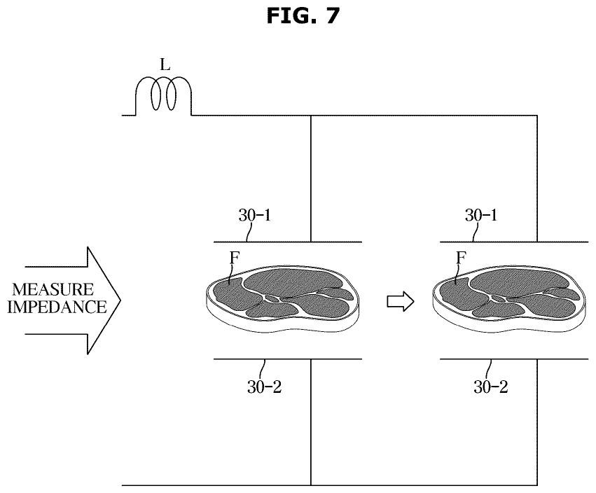

FIG. 7 is a diagram for describing determining the resonance frequency of food a plurality of times according to an embodiment of the disclosure.

Referring to FIGS. 6 and 7, as described above, the resonance frequency may be determined based on the impedances of the food F at each of plurality of frequencies at operation 601.

The at least one processor 22 may determine the resonance frequency at regular intervals to detect changes in the resonance frequency.

That is, the at least one processor 22 may determine the resonance frequency in an initial storage state of the food F, and subsequently continue to determine the resonance frequency of the food at preset intervals. The preset interval may be set as an appropriate interval for determining the state of the food F and may be set differently according to the storage environment of food, and the like.

The at least one processor 22 may determine whether the amount of change in the resonance frequency exceeds a reference value at operation 603. Here, the reference value may be appropriately set as a reference value for identifying whether the food has spoiled.

That is, the initial resonance frequency when the food F was introduced may be compared with the current resonance frequency, and when the difference between the two resonance frequencies exceeds the reference value, it may be determined that the food has spoiled.

In addition, the pH of the food F may be determined based on the resonance frequency, and it may also be determined whether the change in the pH determined at preset intervals exceeds a threshold value, thereby determining whether the food has spoiled.

A table map representing the state of food corresponding to the resonance frequency or pH of food may be stored in the memory 23, and the at least one processor 22 may determine whether food has spoiled based on the table map stored in the memory 23. Details regarding determining whether food has spoiled based on changes in the pH of the food will be described below.

The at least one processor 22 may control the plurality of electrodes 30 to generate a NF electric field for sterilizing food when the amount of change in the resonance frequency exceeds the reference value at operation 605.

As sterilization of the food F is performed by the electric field generated between the plurality of electrodes 30, spoilage of the food may be prevented and long-term storage may be facilitated.

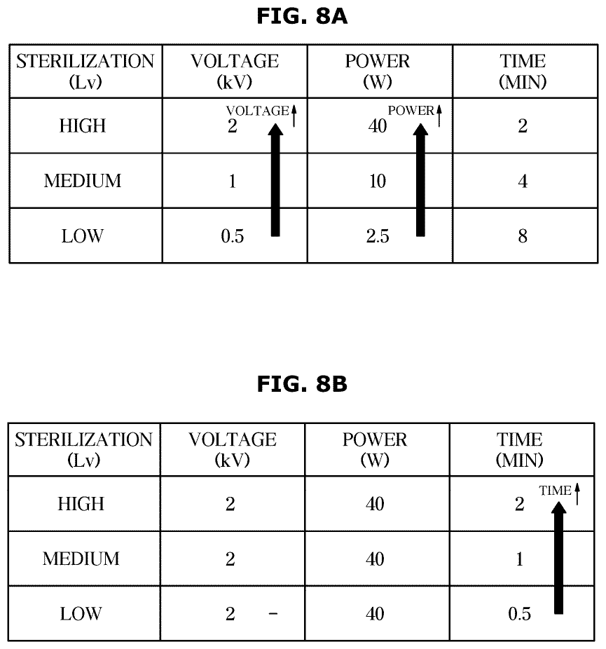

FIG. 8A is a diagram illustrating adjusting a sterilization intensity and time according to an embodiment of the disclosure.

FIG. 8B is a diagram illustrating adjusting a sterilization intensity and time according to an embodiment of the disclosure.

The at least one processor 22 may adjust the intensity of the NF electric fields and the sterilization time according to the degree of food spoilage.

For example, referring to FIG. 8A, the intensity of the NF electric field may be increased by increasing the voltage and power supplied to the plurality of electrodes 30 as the degree of spoilage becomes more severe.

In addition, referring to FIG. 8B, the sterilization time may be increased by increasing a generation time of the NF electric field as the degree of spoilage becomes more severe.

As the intensity of the NF electric field increases or the sterilization time increases, the sterilization strength increases, allowing appropriate sterilization for food with a high degree of spoilage.

Referring to FIGS. 8A and 8B, embodiments of increasing only the sterilization intensity or only the sterilization time are illustrated, but the disclosure is not limited thereto, and various operations for increasing the sterilization strength according to the degree of spoilage, such as simultaneously increasing the sterilization intensity and the sterilization time may be performed.

In embodiments described below, a part-specific degree of spoilage of food may be determined, and in such cases, the direction of the NF electric field may also be controlled to perform focused sterilization on a spoilage region among regions occupied by food. That is, the NF electric field may be intensively emitted toward a region in which food is spoiled, allowing a part-specific sterilization on a spoilage part.

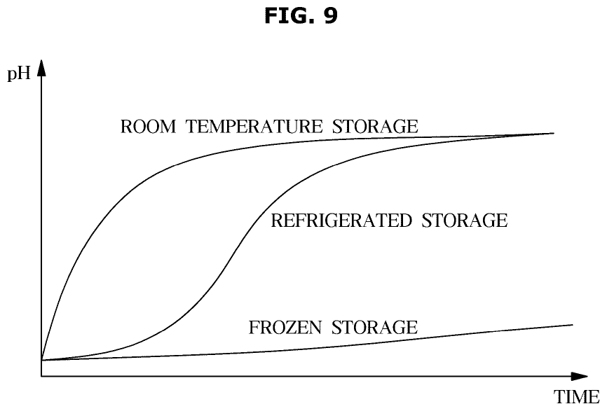

FIG. 9 is a diagram for describing a degree of food spoilage depending on food storage environment according to an embodiment of the disclosure.

Referring to FIG. 9, the at least one processor 22 may determine the resonance frequency at regular intervals to detect changes in the resonance frequency.

That is, the at least one processor 22 may determine the resonance frequency in the initial storage state of the food F, and subsequently continue to determine the resonance frequency of the food at preset intervals.

The preset interval may be set as an appropriate interval for determining the state of the food F and may be set differently according to the storage environment of food, and the like.

For example, at room temperature, food spoilage may proceed relatively rapidly, and in the case of frozen storage, food spoilage may proceed relatively slowly.

The at least one processor 22 may change the preset interval based on such food storage environment.

When food is stored at room temperature, it can be seen that the pH increases relatively rapidly as the storage time elapses as shown in FIG. 9. This indicates that food spoilage proceeds relatively rapidly, and the at least one processor 22 may determine the resonance frequency at relatively shorter intervals considering that food spoilage is fast. That is, the preset interval may be shortened as the storage temperature of food increases.

When food is stored in a frozen environment, it can be seen that the pH increases relatively slowly as the storage time elapses as shown in FIG. 9. This indicates that food spoilage proceeds relatively slowly, and the at least one processor 22 may determine the resonance frequency at relatively longer intervals considering that food spoilage is slow. That is, the preset interval may be lengthened as the storage temperature of food decreases.

When food is stored in a refrigerated environment, the at least one processor 22 may set the preset interval to an intermediate value between the interval for storage in a frozen environment and the interval for storage in a room temperature environment.

By changing the interval for identifying the degree of spoilage according to the food storage environment as described above, appropriate food spoilage identification may be achieved.

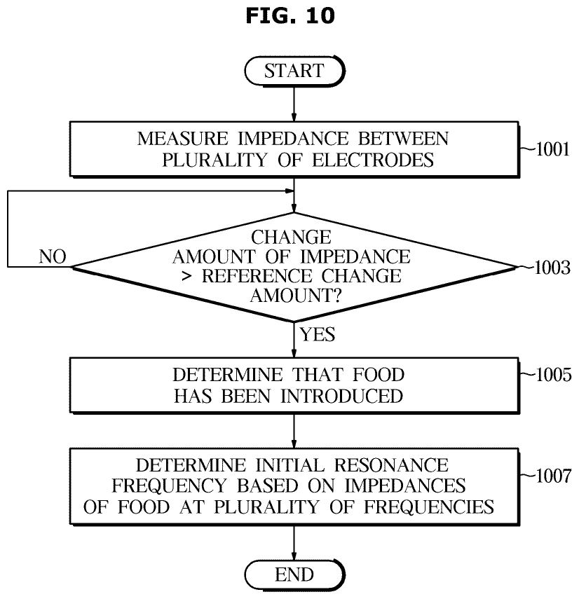

FIG. 10 is a flowchart for describing a process of determining whether food has been introduced according to an embodiment of the disclosure.

As described above, the at least one processor 22 may determine a resonance frequency in an initial storage state of the food F and thereafter compare the resonance frequency with a current resonance frequency to determine the degree of spoilage of the food.

To determine the initial resonance frequency in the initial storage state of food, it is required to first determine whether food has been introduced between the plurality of electrodes 30.

Referring to FIG. 10, based on a change amount of impedance between the plurality of electrodes at operation 1001 being greater than a reference change amount at operation 1003, the at least one processor 22 may determine that food has been introduced at operation 1005.

When no food is introduced between the plurality of electrodes 30, a relatively low impedance value may be detected, and when food is introduced in this state, a sharp increase in the detected impedance may be observed. Therefore, when the change amount of impedance is greater than a reference change amount, it may be determined that food has been introduced.

The at least one processor 22 may, upon determining that food has been introduced, detect impedances of the food at a plurality of frequencies and determine an initial resonance frequency based on the impedances corresponding to the plurality of frequencies at operation 1007.

The initial resonance frequency determined as described above may serve as a reference for comparison with resonance frequencies detected at regular intervals thereafter, to determine whether the food has spoiled.

In addition, as an example of the food management system 1, the at least one processor 22 may determine that food has been introduced by detecting the opening of a door (not shown) included in a food storage apparatus, such as a refrigerator, and then detecting the above-described change in the impedance.

The following describes a process of determining a part-specific resonance frequency of food based on part-specific impedances of the food, and accordingly determining the states of each part (each region) of the food.

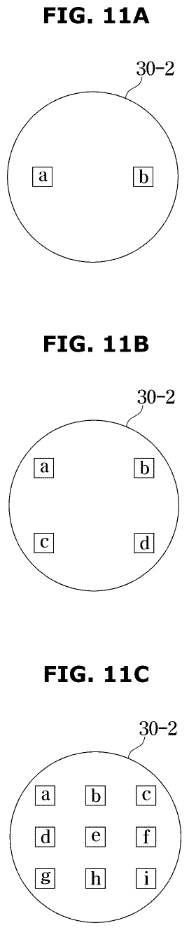

FIGS. 11A to 11C are diagrams illustrating a plurality of sub-electrodes included in each of a plurality of electrodes according to various embodiments of the disclosure.

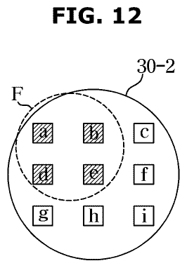

FIG. 12 is a diagram illustrating activation of some of a plurality of sub-electrodes according to an embodiment of the disclosure. and

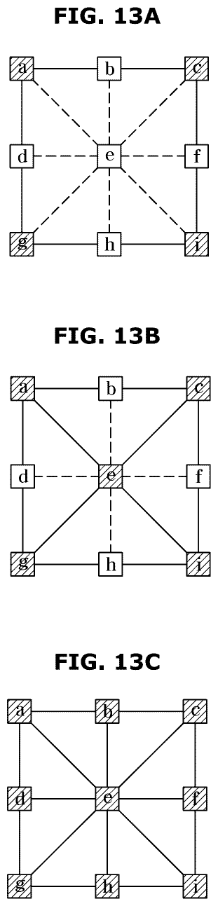

FIGS. 13A, 13B and 13C are diagram illustrating various methods of determining an impedance using a plurality of sub-electrodes according to various embodiments of the disclosure.

Each of the plurality of electrodes 30-1 and 30-2 may include a plurality of sub-electrodes.

Referring to FIGS. 11A to 11C, various numbers of sub-electrodes may be included in each electrode.

Each electrode may include two sub-electrodes 30-2a and 30-2b referring to FIG. 11A, may include nine sub-electrodes 30-2a, 30-2b . . . and 30-2i referring to FIG. 11C. The number and positions of the plurality of sub-electrodes are not limited to those show, and may be provided in various numbers and positions.

When food is placed on the second electrode 30-2, the at least one processor 22 may determine, based on the position of the food, sub-electrodes to be activated from among the plurality of sub-electrodes.

Referring to FIG. 12, when the area occupied by the food is smaller than the area of the second electrode 30-2, the food may be positioned only on some of the plurality of sub-electrodes included in the second electrode 30-2.

In this case, an impedance close to infinity may be detected for sub-electrodes on which the food is not positioned. The at least one processor 22 may activate sub-electrodes excluding the sub-electrodes from which such impedance close to infinity is detected.

Referring to FIG. 12, only some sub-electrodes 30-2a, 30-2b, 30-2d, and 30-2e that are located in the region of the second electrode 30-2 corresponding to the area occupied by the food may be activated.

In addition, the at least one processor 22 may similarly activate only some sub-electrodes 30-1a, 30-1b, 30-1c, and 30-1d for the first electrode 30-1.

Thereafter, the at least one processor 22 may control the frequency modulator 40 such that power at each of plurality of frequencies is sequentially supplied to the activated plurality of sub-electrodes 30-1a, 30-1b, 30-1c, 30-1d, 30-2a, 30-2b, 30-2c, and 30-2d), and determine a region-specific resonance frequency of the food based on a region-specific impedance of the food at each frequency.

By determining a resonance frequency based on the impedance between the activated plurality of sub-electrodes, a region-specific state of the food may be determined based on the region-specific resonance frequency of the food. That is, a region-specific degree of spoilage of the food may be determined.

By determining a region-specific degree of spoilage of the food, a region in which spoilage has occurred may be intensively sterilized, ensuring ease of long-term storage of food.

Referring to FIGS. 13A to 13C, the at least one processor 22 may increase the number of sub-electrodes to be activated according to the degree of spoilage of the food. That is, in the previous description, sub-electrodes served to determine the state of the food, whereas sub-electrodes described with reference to FIGS. 13A to 13C serve to generate an electric field for food sterilization.

In relation to FIGS. 8A and 8B, it has been described that the sterilization intensity and sterilization time may be increased according to the degree of spoilage of the food.

In addition, the sterilization intensity may be increased by increasing the number of sub-electrodes to be activated as the degree of spoilage of the food becomes more severe.

For example, when the degree of spoilage of the food is not severe, only four sub-electrodes 30-2a, 30-2c, 30-2g, and 30-2i may be activated to generate an NF electric field for sterilizing the food as shown in FIG. 13A.

In addition, when the degree of spoilage of the food is severe, all nine sub-electrodes may be activated to generate an NF electric field for food sterilization as shown in FIG. 13C.

By including a plurality of sub-electrodes in each of the plurality of electrodes 30 and activating some or all of the plurality of sub-electrodes, a region-specific degree of spoilage of the food may be detected, and the sterilization strength may be adjusted according to the degree of spoilage of the food.

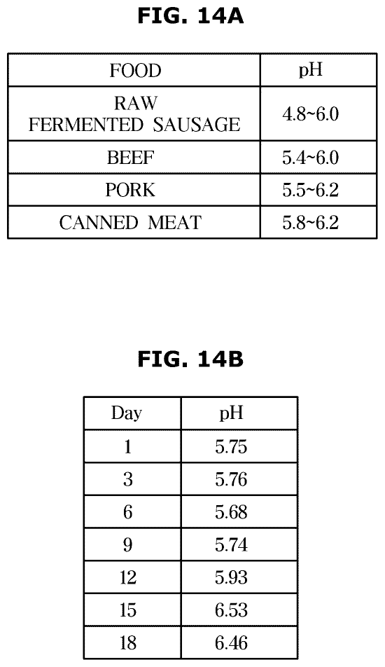

FIGS. 14A and 14B are diagrams for describing normal pH ranges of food and changes in pH over time according to various embodiments of the disclosure.

As described above, the pH of the food F may be determined based on the resonance frequency, and whether food has spoiled may be determined based on the pH of the food.

That is, whether food has spoiled may be determined by determining whether the difference between a pH determined at a preset interval and an initial pH exceeds a reference value.

A table map representing the state of the food corresponding to the pH of the food may be stored in the memory 23, and the at least one processor 22 may determine whether the food has spoiled based on the table map stored in the memory 23.

Referring to FIG. 14A, a normal pH range for each type of food may be stored in the form of a table map.

For example, in the case of “raw fermented sausage”, when the pH falls within a range of 4.8 to 6.0, the food may be determined to be in a normal state without spoilage, and in the case of “beef”, when the pH falls within a range of 5.4 to 6.0, the food may be determined to be in a normal state without spoilage. In the case of “pork”, when the pH falls within a range of 5.5 to 6.2, the food may be determined to be in a normal state without spoilage, and in the case of “canned meat”, when the pH falls within a range of 5.8 to 6.2, the food may be determined to be in a normal state without spoilage.

Since the pH of such food increases as the food spoils, when the pH increases to a level outside the normal range, it may be determined that the food has spoiled.

Referring to FIG. 14B, the change in pH of “beef” over time is illustrated.

From day 1 to day 12 of storage, the pH of the beef remains within a range of 5.4 to 6.0, indicating that the beef is in a normal state without spoilage. However, on day 15 of storage day, the pH of the beef reaches 6.53, which falls outside the normal range, and thus the beef may be determined to be spoiled.

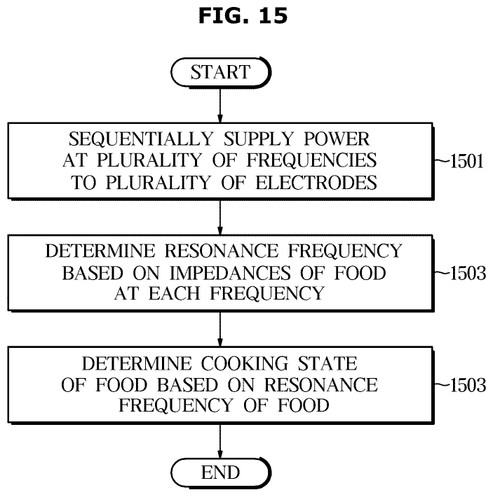

FIG. 15 is a flowchart showing a method of identifying a food cooking state in a food storage system according to an embodiment of the disclosure.

Referring to FIG. 15, the resonance frequency may also be used to identify the degree of cooking or thawing of the food F. The resonance frequency changes as the food F is cooked or thawed. Thus, by periodically measuring such resonance frequency, the degree of cooking or thawing of the food may be determined based on the changes in the resonance frequency.

For this, as described above, the at least one processor 22 may control the frequency modulator 40 such that power at each of a plurality of frequencies is supplied to the plurality of electrodes 30 at operation 1501.

That is, by supplying power at various frequencies, a frequency at which the current or voltage is maximized or the impedance is minimized among the plurality of frequencies may be determined.

Accordingly, the at least one processor 22 may determine the resonance frequency based on the impedances of the food F at each frequency at operation 1503.

The at least one processor 22 may determine the cooking or thawing state of the food F based on the determined resonance frequency of the food at operation 1505.

Specifically, the at least one processor 22 may determine that the cooking or thawing of food is complete based on the resonance frequency of the food not increasing when a cooking or thawing operation is performed. Since the resonance frequency of food increases as the food is cooked or thawed, when the resonance frequency of the food no longer increases, it may be determined that cooking or thawing of the food is complete.

The at least one processor 22 may determine that the cooking or thawing of food is in progress based on the resonance frequency of the food increasing when a cooking operation is performed. Since an increase in the resonance frequency during a cooking or thawing operation indicates that cooking of food is in progress, the at least one processor 22 may adjust the power consumption based on the resonance frequency.

That is, the degree of cooking or thawing of food may be determined based on the resonance frequency, and when cooking or thawing is not properly performed, the power consumption may be increased to increase the intensity of cooking or thawing.

The at least one processor 22 may adjust the power used for thawing or cooking of the food F based on the determined degree of thawing or cooking of the food F to ensure appropriate thawing or cooking.

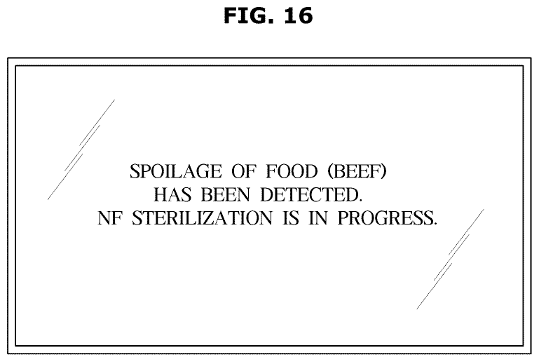

FIG. 16 is a diagram illustrating provision of a notification to a user and the like according to an embodiment of the disclosure.

Referring to FIG. 16, the at least one processor 22 may generate a notification for providing information related to the determined state of the food and an operation corresponding to the state and provide the notification to the user.

For example, when performing a sterilization operation due to food spoilage, the at least one processor 22 may control a separately provided display (not shown) to display a phrase “Spoilage of food (beef) has been detected. NF sterilization is in progress.”

In addition, an auditory notification may be provided to the user through a speaker (not shown), and the like.

Notifications may be generated not only regarding the state of food or a sterilization operation according to the state of food, but also regarding whether food thawing or cooking is complete, or power adjustments based on the degree of thawing or cooking, and the like, and provided to the user.

By receiving such notifications, users may benefit from enhanced convenience regarding food storage, thawing or cooking.

A food management system according to an embodiment may include: a plurality of electrodes arranged to be spaced apart from each other with food therebetween; a frequency modulator configured to adjust a frequency of power supplied to the plurality of electrodes; at least one processor configured to control the plurality of electrodes and the frequency modulator; wherein the at least one processor may be configured to: control the frequency modulator such that power at each of a plurality of frequencies is sequentially supplied to the plurality of electrodes, determine a resonance frequency based on an impedance of the food at each frequency, determine a state of the food based on the resonance frequency of the food, and perform an operation corresponding to the determined state of the food.

The at least one processor may be configured to determine that the food has spoiled when a difference between the resonance frequency of the food and an initial resonance frequency of the food exceeds a reference value.

The at least one processor may be configured to control the plurality of electrodes to generate a near field (NF) electric field for sterilizing the food based on determining that the food has spoiled.

According to the disclosure, the storage period of food may be extended by automatically performing sterilization after identifying the state of food, such as the degree of spoilage.

In addition, as the food state identification and sterilization are automatically performed, user convenience may be enhanced by eliminating the need for users to directly manage food to prevent spoilage.

The at least one processor may be configured to adjust an intensity of the NF electric field and a sterilization time according to a degree to which the food has spoiled.

The at least one processor may be configured to determine the resonance frequency of the food at preset intervals.

The at least one processor may change the preset interval based on a storage environment of the food.

The at least one processor may change the preset cycle to be shorter as the storage temperature of the food increases.

The at least one processor may be configured to determine that the food has been introduced based on a change amount of impedance between the plurality of electrodes being greater than a reference change amount, and determine an initial resonance frequency based on impedances of the food at a plurality of frequencies.

Each of the plurality of electrodes may include a plurality of sub-electrodes, and the at least one processor may be configured to determine, based on a position of the food, sub-electrodes to be activated from among the plurality of sub-electrodes, control the frequency modulator such that power at each of a plurality of frequencies is sequentially supplied to the activated sub-electrodes, determine a region-specific resonance frequency of the food based on a region-specific impedance of the food at each frequency, and determine a region-specific state of the food based on the region-specific resonance frequency of the food.

The at least one processor may be configured to determine a pH value of the food based on the determined resonance frequency and determine the state of the food based on the determined pH value.

The at least one processor may be configured to determine a cooking or thawing state of the food based on a change in the resonance frequency of the food.

The at least one processor may be configured to: determine that cooking or thawing of the food is in progress based on an increase in the resonance frequency of the food, and adjust power consumption based on the resonance frequency of the food

According to the disclosure, appropriate cooking may be achieved by determining a cooking state of food, such as the degree of thawing, and adjusting power accordingly.

The at least one processor may be configured to determine that the cooking or thawing of the food is complete based on an increase in the resonance frequency of the food being less than a reference increase amount.

The at least one processor may generate a notification to provide information related to the determined state of the food and an operation corresponding to the state.

A method of controlling a food management system according to an embodiment including a plurality of electrodes arranged to be spaced apart from each other with food therebetween and a frequency modulator configured to adjust a frequency of power supplied to the plurality of electrodes may include: controlling the frequency modulator such that power at each of a plurality of frequencies is sequentially supplied to the plurality of electrodes; determining a resonance frequency based on an impedance of the food at each frequency; determining a state of the food based on the resonance frequency of the food; and performing an operation corresponding to the determined state of the food.

The determining of the state of the food may include determining that the food has spoiled when a difference between the resonance frequency of the food and an initial resonance frequency of the food exceeds a reference value.

The method may further include controlling the plurality of electrodes to generate a near field (NF) electric field for sterilizing the food based on determining that the food has spoiled.

The sterilizing of the food may include adjusting an intensity of the NF electric field and a sterilization time according to a degree to which the food has spoiled.

The determining of the resonance frequency may include determining the resonance frequency of the food at preset intervals.

The method may further include changing the preset interval based on a storage environment of the food.

The changing of the preset interval may include shortening the preset interval as a storage temperature of the food increases.

The determining of the state of the food may include determining that the food has been introduced based on a change amount of impedance between the plurality of electrodes being greater than a reference change amount, and determining an initial resonance frequency based on impedances of the food at a plurality of frequencies.

Each of the plurality of electrodes may include a plurality of sub-electrodes, and the determining of the state of the food may include determining, based on a position of the food, sub-electrodes to be activated from among the plurality of sub-electrodes, controlling the frequency modulator such that power at each of a plurality of frequencies is sequentially supplied to the activated sub-electrodes, determining a region-specific resonance frequency of the food based on a region-specific impedance of the food at each frequency, and determining a region-specific state of the food based on the region-specific resonance frequency of the food.

The determining of the state of the food may include determining a pH value of the food based on the determined resonance frequency and determining the state of the food based on the determined pH value.

The determining of the state of the food may include determining a cooking or thawing state of the food based on a change in the resonance frequency of the food.

The determining of a cooking or thawing state of the food may include determining that cooking or thawing of the food is in progress based on an increase in the resonance frequency of the food, and adjusting power consumption based on the resonance frequency of the food.

The determining the cooking or thawing state of the food may include determining that cooking or thawing of the food is complete based on an increase in the resonant frequency of the food being less than a reference increase amount.

The method may further include generating a notification to provide information related to the determined state of the food and an operation corresponding to the state.

According to an aspect of the disclosure, the storage period of food may be extended by automatically performing sterilization after identifying the state of food, such as the degree of spoilage.

In addition, as the food state identification and sterilization are automatically performed, user convenience may be enhanced by eliminating the need for users to directly manage food to prevent spoilage.

According to an aspect of the disclosure, appropriate cooking may be achieved by determining a cooking state of food, such as the degree of thawing, and adjusting power accordingly.

Meanwhile, the disclosed embodiments may be embodied in the form of a recording medium storing instructions executable by a computer. The instructions may be stored in the form of program code and, when executed by a processor, may generate a program module to perform the operations of the disclosed embodiments. The recording medium may be embodied as a computer-readable recording medium.

The computer-readable recording medium includes all kinds of recording media in which instructions which may be decoded by a computer are stored, for example, Read Only Memory (ROM), Random Access Memory (RAM), a magnetic tape, a magnetic disk, flash memory, an optical data storage device, and the like.

While the disclosure has been shown and described with reference to various embodiments thereof, it will be understood by those skilled in the art that various changes in form and details may be made therein without departing from the spirit and scope of the disclosure as defined by the appended claims and their equivalents.

What is claimed is:

1. A food management system comprising:
a plurality of electrodes arranged to be spaced apart from each other with food therebetween;
a frequency modulator configured to adjust a frequency of power supplied to the plurality of electrodes;
memory, comprising one or more storage media, storing instructions; and
at least one processor communicatively coupled the plurality of electrodes, the frequency modulator and the memory,
wherein the instructions, when executed by the at least one processor individually or collectively, cause the food management system to:
control the frequency modulator such that power at each of a plurality of frequencies is sequentially supplied to the plurality of electrodes,
determine a resonance frequency based on an impedance of the food at each frequency,
determine a state of the food based on the resonance frequency of the food, and
perform an operation corresponding to the determined state of the food.

2. The food management system of claim 1, wherein the instructions, when executed by the at least one processor individually or collectively, further cause the food management system to determine that the food has spoiled when a difference between the resonance frequency of the food and an initial resonance frequency of the food exceeds a reference value.

3. The food management system of claim 1, wherein the instructions, when executed by the at least one processor individually or collectively cause the food management system to control the plurality of electrodes to generate a near field (NF) electric field for sterilizing the food based on determining that the food has spoiled.

4. The food management system of claim 3, wherein the instructions, when executed by the at least one processor individually or collectively, further cause the food management system to adjust an intensity of the NF electric field and a sterilization time according to a degree to which the food has spoiled.

5. The food management system of claim 1, wherein the instructions, when executed by the at least one processor individually or collectively, further cause the food management system to determine the resonance frequency of the food at preset intervals, and shorten the preset interval as a storage temperature of the food increases.

6. The food management system of claim 2, wherein the instructions, when executed by the at least one processor individually or collectively, further cause the food management system to determine that the food has been introduced based on a change amount of impedance between the plurality of electrodes being greater than a reference change amount, and determine an initial resonance frequency based on impedances of the food at a plurality of frequencies.

7. The food management system of claim 1,
wherein each of the plurality of electrodes includes a plurality of sub-electrodes, and
wherein the instructions, when executed by the at least one processor individually or collectively, further cause the food management system to:
determine, based on a position of the food, sub-electrodes to be activated from among the plurality of sub-electrodes,
control the frequency modulator such that power at each of a plurality of frequencies is sequentially supplied to the activated sub-electrodes,
determine a region-specific resonance frequency of the food based on a region-specific impedance of the food at each frequency, and
determine a region-specific state of the food based on the region-specific resonance frequency of the food.

8. The food management system of claim 1, wherein the instructions, when executed by the at least one processor individually or collectively, further cause the food management system to determine a pH value of the food based on the determined resonance frequency and determine the state of the food based on the determined pH value.

9. The food management system of claim 1, wherein the instructions, when executed by the at least one processor individually or collectively, further cause the food management system to determine a cooking or thawing state of the food based on a change in the resonance frequency of the food.

10. The food management system of claim 9, wherein the instructions, when executed by the at least one processor individually or collectively, further cause the food management system to:
determine that cooking or thawing of the food is in progress based on an increase in the resonance frequency of the food, and adjust power consumption based on the resonance frequency of the food; and
determine that the cooking or thawing of the food is complete based on an increase in the resonance frequency of the food being less than a reference increase amount.

11. A method of controlling a food management system including a plurality of electrodes arranged to be spaced apart from each other with food therebetween and a frequency modulator configured to adjust a frequency of power supplied to the plurality of electrodes, the method comprising:
controlling the frequency modulator such that power at each of a plurality of frequencies is sequentially supplied to the plurality of electrodes;
determining a resonance frequency based on an impedance of the food at each frequency;
determining a state of the food based on the resonance frequency of the food; and
performing an operation corresponding to the determined state of the food.

12. The method of claim 11, wherein the determining of the state of the food includes determining that the food has spoiled when a difference between the resonance frequency of the food and an initial resonance frequency of the food exceeds a reference value.

13. The method of claim 11, further comprising controlling the plurality of electrodes to generate a near field (NF) electric field for sterilizing the food based on determining that the food has spoiled.

14. The method of claim 13, wherein the sterilizing of the food includes adjusting an intensity of the NF electric field and a sterilization time according to a degree to which the food has spoiled.

15. The method of claim 11,
wherein the determining of the resonance frequency includes determining the resonance frequency of the food at preset intervals, and
wherein shortening the preset interval as a storage temperature of the food increases.

16. The method of claim 12, further comprising:
determining that the food has been introduced based on a change amount of impedance between the plurality of electrodes being greater than a reference change amount; and
determining an initial resonance frequency based on impedances of the food at a plurality of frequencies.

17. The method of claim 12, wherein the determining of whether the food has spoiled includes determining whether the food has spoiled based on a table map stored in memory.

18. The method of claim 17, wherein the table map includes a state of the food corresponding to a resonance frequency of the food or a pH of the food.

19. One or more non-transitory computer-readable storage media storing one or more computer programs including computer-executable instructions that, when executed by one or more processors of an electronic device individually or collectively, cause the electronic device to perform operations, the electronic device communicative coupled to a plurality of electrodes arranged to be spaced apart from each other with food therebetween and a frequency modulator configured to adjust a frequency of power supplied to the plurality of electrodes, the operations comprising:
controlling, by the electronic device, the frequency modulator such that power at each of a plurality of frequencies is sequentially supplied to the plurality of electrodes;
determining, by the electronic device, a resonance frequency based on an impedance of the food at each frequency;
determining, by the electronic device, a state of the food based on the resonance frequency of the food; and
performing, by the electronic device, an operation corresponding to the determined state of the food.

20. The one or more non-transitory computer-readable storage media of claim 19, wherein the determining of the state of the food includes determining that the food has spoiled when a difference between the resonance frequency of the food and an initial resonance frequency of the food exceeds a reference value.
